(19) United States
(12) Patent Application Publication
(10) Pub. No.: US 2004/0059195 A1
(43) Pub. Date: Mar. 25, 2004

(54) Device for attaching equipment to an appendage

(76) Inventor: Ken W. Ridenour, Amarillo, TX (US)

Correspondence Address: HOWREY SIMON ARNOLD & WHITE LLP, 750 BERING DRIVE, HOUSTON, TX, 77057, US

(21) Appl. No.: 10/252,267

(22) Filed: Sep. 23, 2002

(51) Int. Cl. A61B 5/00

(52) U.S. Cl. 600/300

(57) ABSTRACT

The health and behaviors of feedlot animals may be observed with monitoring and administering equipment or electronics. The equipment or electronics is contained in an attachment device, which attaches to an appendage of the feedlot animals. The device is closed about the appendage and may be further clamped on the appendage. A fastening mechanism holds the device closed on the appendage. An end piece on the device may bend a flexible appendage, such as a tail, to help hold the device on the appendage. A series of studs within the device may be provided to hold the device on the appendage. The device may also include a frusto-conical bore to accommodate the appendage and help hold the device thereon.

### FIELD OF THE INVENTION

The present invention relates generally to a monitoring system for animals and, more particularly to a device for attaching monitoring or administering equipment or electronics to an appendage of an animal.

### BACKGROUND OF THE INVENTION

In the cattle business and the feedlot industry in particular, as many as forty to fifty thousand head of cattle may be located at any given time in a typical feedlot. Each animal may have an average pen space of 150-250 square feet. When animals are maintained in such close quarters, it is necessary to diligently monitor the health of the animals so that an animal can be removed and treated as soon as is shows symptoms of illness. Traditionally, feedlots have a person who rides on a horse into the individual pens and looks for animals that appear lethargic or demonstrate some other characteristic of illness or poor health. As can be expected, this system is only as good as the person's ability to observe such characteristics. Furthermore, the characteristics of illness in the animal may only become visible after the illness has substantially progressed.

Behaviors of an animal, such as the frequency of the animal's intake of food and water, and physiological parameters, such as the temperature and pulse rate of the animal, can be good indicators of the animal's health and well being. Timely monitoring of certain behaviors or physiological parameters of each animal to identify the early stages of illness prior to the onset of clinical signs would be a tremendous economic benefit to an animal owner.

Various patents have focused on monitoring and measuring the feed consumption and weight change of animals. For example, U.S. Pat. Nos. 3,465,724; 3,541,995; 3,929,277; 4,532,892; 4,617,876, disclose devices to monitor or control the feeding times or amounts consumed by the animal. Other patents attempt to monitor an animal's temperature and detect fever or illness. Some prior art monitoring devices implant in the animal or insert into a body cavity. For example, U.S. Pat. Nos. 3,781,837; 3,893,111; 4,399,821; 4,854,328; and 4,865,044 disclose devices that are inserted into a body cavity or implanted in the animal.

Alternatively, a non-intrusive, prior art monitoring device is disclosed in U.S. Pat. No. 6,113,539 and is illustrated in FIG. 1. The non-intrusive monitoring device includes a sleeve 10 having an outer surface 12, an interior 14, and an inner surface 16. The monitoring sleeve 10 has a pouch 20 to house a separate and insertable instrument pack 22. The pouch 20 is located on the outer surface 12 of the sleeve 10. One or more biosensors 40 are affixed to the inner surface 16 of the monitoring sleeve 10. The biosensors 40 are connected to a data port on the instrument pack 22 via electrical wires 42 embedded within the interior 14 of the sleeve 10. The sleeve 10 is wrapped around the tail of the animal to be monitored so that the biosensor 40 is placed adjacent the skin of the animal. Fasteners 30 and 32, such as Velcro™, are then used to hold the sleeve 10 on the tail.

Another non-intrusive monitoring device is also disclosed in U.S. Pat. No. 6,113,539 and is illustrated in FIG. 2. The monitoring device includes a monitoring sleeve 50 having a bottom portion 60 and a top portion 70. The bottom portion 60 is shaped like a shallow, rounded “W.” This bottom portion 60 is made of plastic and is slightly flexible. One or more biosensors 82 are located on the inner surface 64 of the sleeve at the apex 66 of this “W”-shaped bottom portion 60. The top portion 70 of the sleeve 50 is comprised of a stretchable fabric, such as elastic, that allows it to be stretched snugly around the animal's tail. On the top portion of the sleeve 70 is a pouch 80 and light 84. The top portion 70 is affixed to the bottom portion 60 by strips of Velcro™ or another fastening mechanism 62 and 72. Both the bottom and top portions 60 and 70 of the sleeve 50 have a rubbery coating on their inner surfaces 64 and 74. This coating helps prevent the sleeve from sliding off or rotating around the animal's tail.

Although the monitoring sleeves 10 and 50 disclosed in U.S. Pat. No. 6,113,539 are effective, those in the art are continually striving to improve ways to monitor animals. In one aspect, for example, the sleeves 10 and 50 can be limited by the type and weight of electronics and equipment that they can successfully hold on the animal. The present invention is directed to an improved device for attaching monitoring or administering equipment or electronics to an appendage of an animal and is directed to overcoming, or at least reducing the effects of, one or more of the problems set forth above.

### SUMMARY OF THE INVENTION

The present invention provides a device for attaching equipment or electronics to an appendage of an animal. The device includes one portion hingedly attached to another portion. The equipment or electronics is disposed in a cavity defined in one of the portions. The equipment or electronics can include monitoring or administering systems. The device attaches to an appendage of an animal by enclosing the appendage within the two portions of the device. The two portions are fastened or bound together to hold the device on the appendage. The device can include an end piece, which bends the appendage, such as a tail, and helps hold the device on the appendage. The device can include a series of studs within the two portions to help hold the device on the appendage. The device can also include a frusto-conical bore to accommodate the appendage and help hold the device thereon.

The foregoing summary is not intended to summarize each potential embodiment or every aspect of the invention disclosed herein, but merely to provide a general summary of the disclosed invention.

### BRIEF DESCRIPTION OF THE DRAWINGS

The foregoing summary, a preferred embodiment, and other aspects of the present invention will be best understood with reference to a detailed description of specific embodiments of the invention, which follows, when read in conjunction with the accompanying drawings, in which:

FIG. 1 illustrates an animal monitoring system according to the prior art.

FIG. 2 illustrates another animal monitoring system according to the prior art.

FIGS. 3A-D illustrate various views of an embodiment of an attachment device according to the present invention.

FIGS. 4A-B illustrate various views of another embodiment of an attachment device according to the present invention.

FIG. 5 illustrates an exemplary location on an animal for the attachment device of FIGS. 4A-B.

FIGS. 6A-D illustrate various views of a first portion of the attachment device of FIGS. 4A-B.

FIGS. 7A-B illustrate various views of a second portion of the attachment device of FIGS. 4A-B.

FIGS. 8A-E illustrate components of embodiments of fastening or binding mechanisms in accordance with the present invention.

FIGS. 9A-C illustrate embodiments of electronics and equipment for use with an attachment device in accordance with the present invention.

FIG. 10 illustrates a cross-sectional view of yet another embodiment of an attachment device according to the present invention.

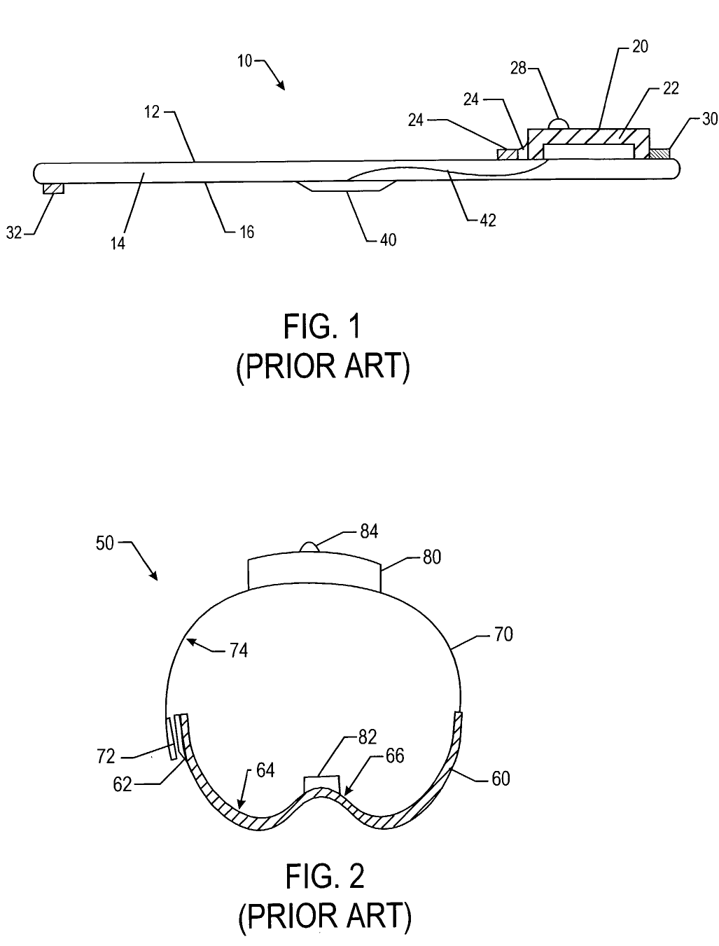

While the invention is susceptible to various modifications and alternative forms, specific embodiments have been shown by way of example in the drawings and will be described in detail herein. However, it should be understood that the invention is not intended to be limited to the particular forms disclosed. Rather, the invention is to cover all modifications, equivalents and alternatives falling within the scope of the invention as defined by the appended claims.

### DETAILED DESCRIPTION OF THE INVENTION

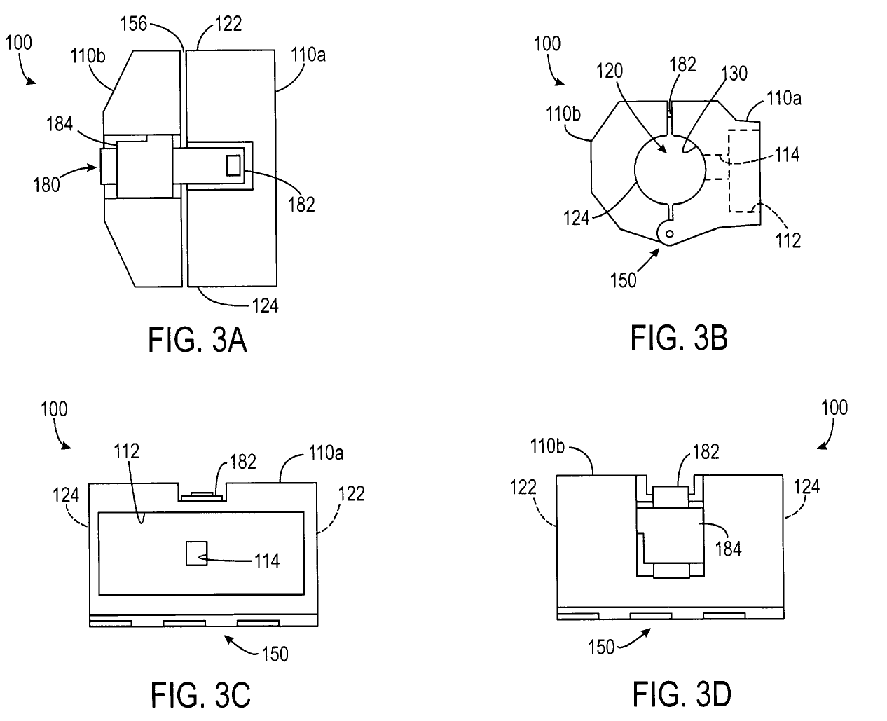

Referring to FIGS. 3A-D, an embodiment of an attachment device 100 according to the present invention is illustrated in a number of views. FIG. 3A illustrates a top view of the attachment device 100; FIG. 3B illustrates an end view of the attachment device 100; FIG. 3C illustrates a side view of the attachment device 100; and FIG. 3D illustrates another side view of the attachment device 100.

The attachment device 100 includes a first body portion 110a, a second body portion 110b, a hinge mechanism 150, and a fastening or binding mechanism 180. The body portions 110a and 110b are attached together along adjacent edges by the hinge mechanism 150, which allows the body portions 110a and 110b to pivot in relation to one about the hinge mechanism 150. Although the present embodiment only has two body portions 110a and 110b, it is understood that the attachment device 100 of the present invention can include more than two body portions. For example, the attachment device of the present invention can include a middle body portion hingedly connected to two side body portions by hinge mechanisms.

The body portions 110a and 110b define an internal opening or bore 120 accommodating an appendage (not shown). The internal opening or bore 120 has first and second ends 122 and 124 and includes a surface 130 to engage or grip the appendage. In one embodiment, the device 100 is attached to a tail of an animal. It is understood that the device 100 can be attached to another appendage of the animal, such as a leg. Moreover, it is understood that the device 100 can be attached to a stalk or branch of a plant requiring attachment of monitoring or administrating equipment or electronics.

In one embodiment, the surface 130 includes a series of studs (not shown), which project from the inner surface of the internal bore 120 to grip the appendage. In other embodiments, the surface 130 can include other surfaces known in the art for making frictional contact with hair or skin of an animal. For example, the surface 130 can include, but is not limited to, a rough surface, a comb structure, or a rubbery surface, among other possibilities known in the art.

Connected by the hinge mechanism 150, the first and second body portions 110a and 110b can be opened and closed in relation to one another. Closing the body portions 110a and 110b decreases the dimension of the bore 120 defined therebetween and encloses the device 100 on the appendage disposed in the bore 120. In one embodiment, the hinge mechanism 150 includes a plurality of inter-fitting knuckles having a pivot pin passing therethrough about which the knuckles rotate. In other embodiments, the hinge mechanism 150 can include mechanisms known in the art for hingedly attaching portions of a body together. For example, the hinge mechanism 150 can include, but is not limited to, a flexible member or portion connecting the body portions 110a and 110b. Alternatively, the hinge mechanism 150 can include a piece of material having a flexible fold therein and integrally connected to the body portions 110a and 110b.

When the body portions 110a and 110b are enclosed about the appendage disposed within the bore 120, the fastening or binding mechanism 180 fastens the body portions 110a and 110b together. It is understood that the fastening or binding mechanism 180 can include a number of devices known in the art for attaching, fastening, or binding hingedly attached portions of a body together. For example, the fastening or binding mechanism 180 can include, but is not limited to, straps, belts, buckles, Velcro™ strips, elastic bands, laces, hooks, loops, latches, clasps, or hasps, among other mechanisms known in the art.

In one embodiment, the fastening or binding mechanism 180 includes a first member 182 attached to the first body portion 110a and includes a second member 184 attached to the second body portion 110b. The first and second members 182 and 184 connect with one another to fasten the body portions 110a and 110b together about the appendage. In a preferred embodiment, the fastening or binding mechanism 180 can be used to clamp or squeeze the body portions 110a and 110b about the appendage. Furthermore, the fastening or binding mechanism 180 is preferably adjustable, allowing the device 100 to be fastened about various sized appendages and allowing the degree of fastening on the appendage to be adjusted.

The device 100 of the present invention holds equipment or electronics (not shown) on the appendage, such as on an animal's tail. The device 100 can include a pouch or pocket receiving the equipment and electronics, which can be a self-contained instrument pack. In addition, the equipment or electronics in the form of a self-contained component or unit can attach to the outside of the device 100. It is understood that the equipment or electronics can be attached to, housed in, or held on the device 100 in a myriad of ways or by a number of mechanisms known in the art.

Preferably, the device 100 offers some form of protection for the equipment and electronics from the weather, physical hazards, etc. In a preferred embodiment, the first body portion 110a defines a cavity 112 for holding the equipment or electronics. The second body portion 110b can just as easily define the cavity or can also define a second cavity in addition to the first cavity 112. A cover (not shown) encloses the equipment or electronics within the cavity 112. A passage or aperture 114 communicates the cavity 112 with the bore 120. Sensors, intravenous lines, or other equipment or electronics (not shown) are positioned adjacent the appendage disposed within the bore 120 and are connected through the passage 114 with the equipment or electronics housed in the cavity 112.

As will be discussed in more detail below, the attachment device 100 can be used in combination with equipment or electronics for measuring or monitoring physiological parameters, such as an animal's temperature or pulse rate. The device 100 can also be used in combination with equipment or electronics for measuring or monitoring an animal's movements, eating habits, or location, among other possible uses. For example, the attachment device 100 of the present invention can be used in combination with monitoring systems and methods as disclosed in U.S. Pat. No. 6,113,539, which is incorporated herein by reference in its entirety. In addition, the attachment device 100 can be used in combination with equipment or electronics for administering substances to an animal, such as injections of antibiotics, vaccines, steroids, hormones, or medicines.

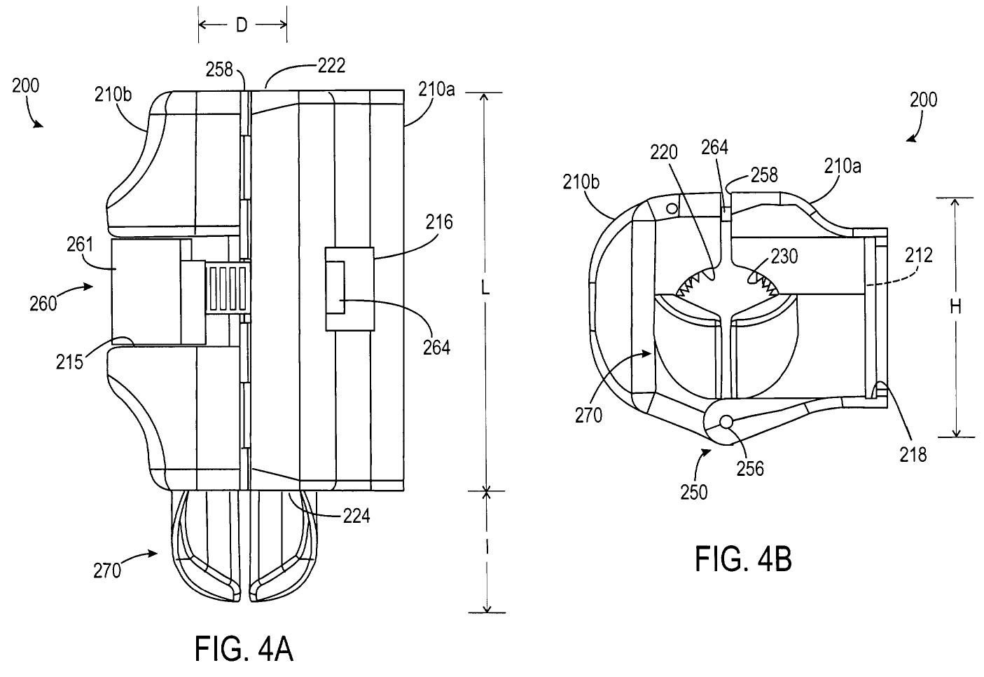

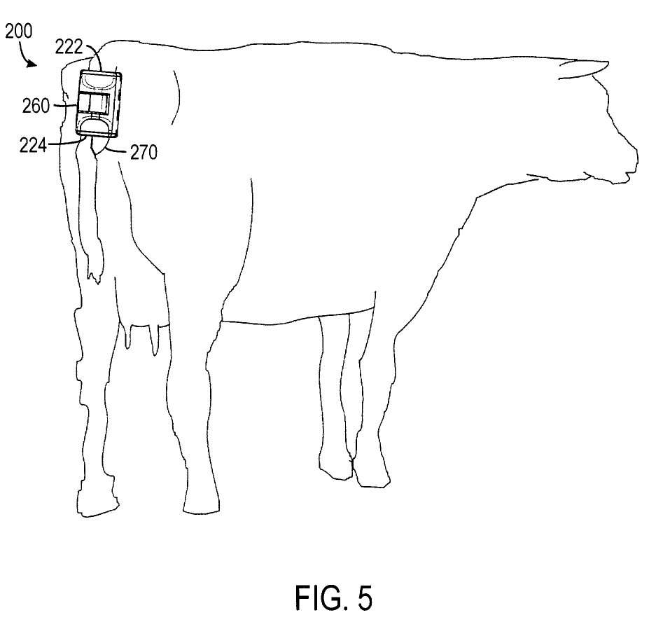

Referring to FIGS. 4A-B, another embodiment of an attachment device 200 according to the present invention is illustrated. FIG. 4A illustrates a top view of the attachment device 200; and FIG. 4B illustrates an end view of the attachment device 200. The attachment device 200 includes a first body portion 210a, a second body portion 210b, a hinge mechanism 250, a fastening or binding mechanism 260, and an end piece or tail piece 270.

The body portions 210a and 210b are formed of rigid material and are attached together along adjacent edges by the hinge mechanism 250, which allows the body portions 210a and 210b to pivot in relation to one. The body portions 210a and 210b define an internal opening or bore 220 accommodating an appendage (not shown), such as a tail of an animal. The internal opening or bore 220 has first and second ends 222 and 224 and includes a surface 230 to engage or grip the appendage. As described in more detail below, the surface 230 includes a series of studs projecting from the inner surface of the internal bore 220. The first body portion 210a includes a cavity 212 defined therein that is covered by a cover or plate (not shown). The cavity 212 holds equipment or electronics as disclosed herein and communicates with the bore 220, and hence the appendage, through a passage (not shown).

Connected by the hinge mechanism 250, the body portions 210a and 210b can be opened and closed in relation to one another. Closing the body portions 210a and 210b decreases the dimension of the bore 220 defined therebetween and encloses the device 200 on the appendage disposed in the bore 220. As described in more detail below, the hinge mechanism 250 includes a plurality of inter-fitting knuckles and having a pivot pin 256 passing therethrough about which the knuckles rotate.

The hinge mechanism 250 preferably connects the body portions 210a and 210b together such that a gap 258 is present along the unconnected edges of the portions 210a and 210b. As best shown in FIG. 4B, the gap 258 is present between the body portions 210a and 210b when they are positioned parallel to one another. Consequently, the fastening or binding mechanism 260 can bring the free edges of the body portions 210a and 210b closer together to further decrease the dimension of the bore 220 about the appendage disposed therein. In this way, the attachment device 200 can clamp or squeeze the gripping surface 230 of the bore 220 against the appendage.

Additionally, the hinge mechanism 250 can be biased or spring loaded to maintain the body portions 110a and 110b in a closed position. The biased or spring loaded hinge mechanism 250 can further help maintain the device 200 on the appendage. Moreover, the biased or spring loaded hinge mechanism 250 can facilitate the attachment and removal of the device 200 by allowing the device 200 to be temporary held on the appendage while the fastening mechanism 260 is operated.

The fastening or binding mechanism 260 fastens body portion 210a and 210b together once the device 200 is enclosed about the appendage disposed within the bore 220. In a preferred embodiment, the fastening mechanism 260 is able to clamp or squeeze the body portions 210a and 210b about the appendage. Furthermore, the fastening mechanism 260 is preferably adjustable, allowing the device 200 to be fastened about various sized appendages and allowing the degree of fastening to be adjusted.

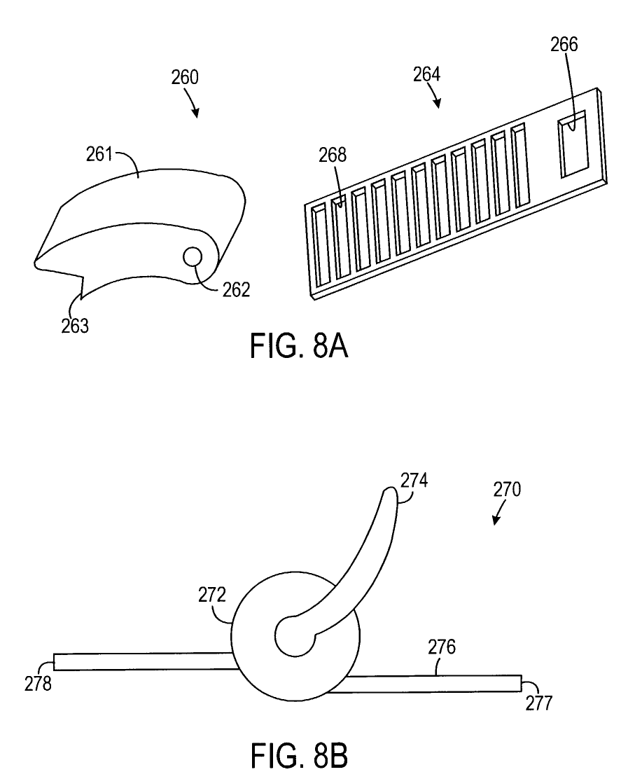

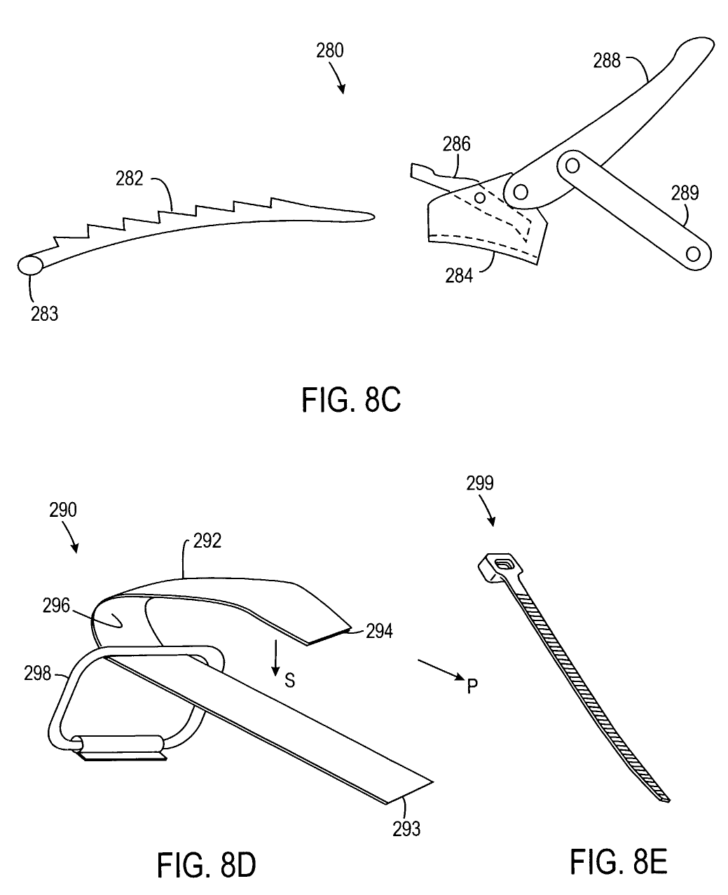

In the present embodiment, the fastening or binding mechanism 260 includes a latch or hasp 261 and an interconnecting member or strap 264, which are shown in relevant detail in FIG. 8A. The latch or hasp 261 is disposed in a well 215 defined in the second body portion 210b and is hingedly attached to the sides of the well 215. One end of the interconnecting member 264 is disposed through an opening 216 and is attached to the first body portion 210b. The interconnecting member 264 includes a plurality of notches or slots that connect with the latch or hasp 261 and that allow for adjustment of the fastening mechanism 260.

The end piece or tailpiece 270 is attached to the device 200 adjacent the second end 224 of the bore 220. The end piece 270 is curved to produce a bend or kink in a flexible portion of the appendage extending from the second end 224. The bending of a flexible appendage, such as a tail, further helps to hold the device 200 on the appendage.

Referring to FIG. 5, a preferred location for the attachment device 200 of FIGS. 4A-B on an animal is illustrated. In FIG. 5, a cow is used as an exemplary animal on which to attach the device 200. It is understood that the present invention is not limited to attachment to cattle, but can be used on other animals. The preferred location of the device 200 for cattle is around the upper end of the tail. The tail is easily accessible and small in diameter, allowing the device 200 to be small and simple to attach or remove.

The device 200 is fastened on the tail with the first end 222 adjacent the base of the tail. The end piece 270 adjacent the second end 224 bends the extending portion of the tail away from the animal and aids in holding the device 200 on the tail. The fastening mechanism 260 and cavity are readily accessible by an operator while the device 200 is attached to the tail.

With the small device 200 located at the base of the tail, the animal can still use the tail to ward away insects. Because the upper end of the tail folds over the rear of the animal, near the anus, biosensors within the device 200 can yield accurate temperature measurements. The biosensors are not inserted into a body cavity or into an incision, which eliminates the possibility of the biosensors being dislodged or expelled and eliminates the chance of an infection occurring at an incision site.

Returning to FIGS. 4A-B, example dimensions of the attachment device 200 for use on the tail of cattle will be discussed. The body portions 210a and 210b have an exemplary length (L) of approximately 4.5 inches and an exemplary height (H) of approximately 2.5 inches. When the device 200 is closed, a general diameter (D) of the bore 220 is roughly 1.5 inches on the first end 222 adjacent the base of the tail. The end piece 270 extends a length (l) of approximately 1.24-inch from the second end 224 of the bore 120 and has an approximately 60-degree up turn. It is understood that the approximate dimensions disclosed herein are only exemplary and will vary for specific implementations of the attachment device 200.

The attachment device 200 is preferably composed of a composite plastic to maintain a light weight and to resist a corrosive environment. The device 200 can weigh as little as one pound, for example, with equipment or electronic components installed in the cavity 212. The body portions 210a and 210b can be separately formed by an injection molding process and then hingedly attached to one another. The studs of the gripping surface 230 can be integrally formed with the body portions 210a and 210b. Alternatively, the studs of the gripping surface 230 can be composed of a different material from the body portions, such as rubber, affixed to the inner surface of the bore 220.

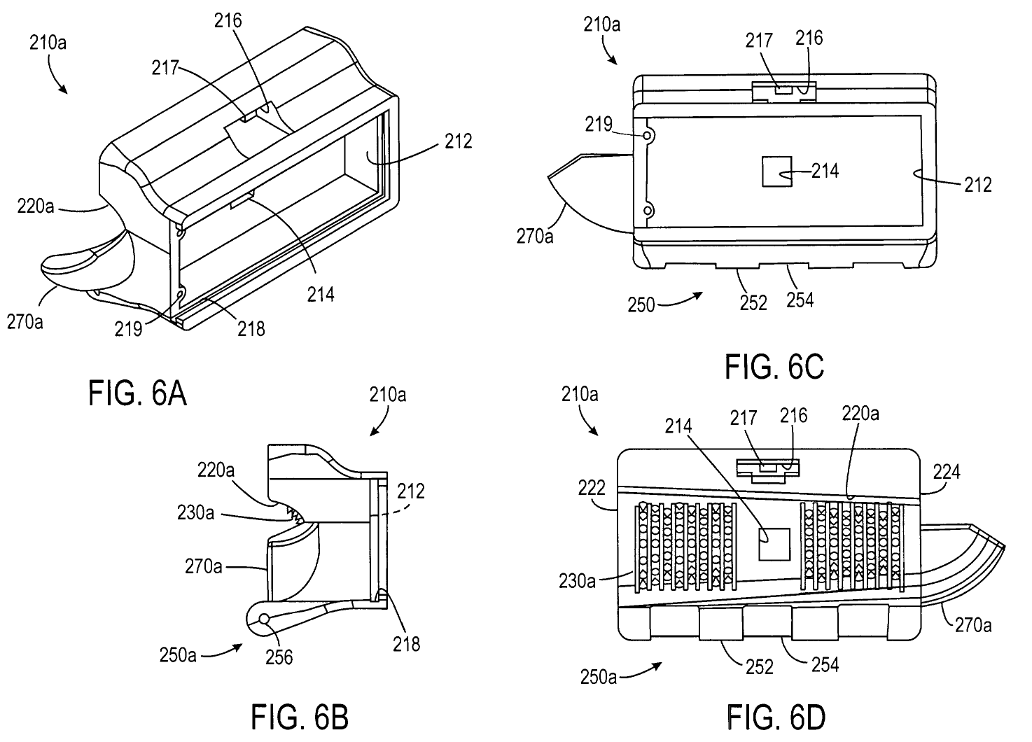

Referring to FIGS. 6A-D, the first body portion 210a of the attachment device is illustrated in a perspective view, an end view, a first side view, and a second side view, respectively. As best shown in FIGS. 6A and 6C, the first body portion 210a defines the cavity 212 for holding equipment or electronics (not shown). The cavity 212 communicates with the internal bore 220a through the passage 214 defined in the body portion 210a.

With the present availability of small electronic and mechanical components, the cavity 212 need not be deeply inset into the body portion 210a. For example, using the exemplary dimensions discussed above, the cavity 212 can be inset approximately 0.92 of an inch into the body portion. The cavity 212 can have a width of approximately 1.6-inches and can have a length of almost 4.5-inches. Thus, the cavity 212 can provide about 6.6-cubic inches of space to house the electronics and equipment.

A cover or plate (not shown) positions over the cavity 212 to protect the equipment or electronics therein. The cover is inserted into a track 218 from the second end 224 of the body portion 210a and is slid along the track 218 to cover the cavity 212. The cover is then affixed to apertures 219 with fasteners (not shown). The track 218 and/or cover can include a seal (not shown) to sealably protect the electronics and equipment. The cavity 212 and cover protect the equipment and electronics from the weather, physical hazards, etc. Furthermore, the cavity 212 and cover are accessible from outside the body portion 210a, permitting an operator to access the equipment or electronics without removing the attachment device from the appendage.

The first body portion 210a defines an opening 216 for use with a fastening or binding mechanism (not shown). The first body portion 210a includes a series of hinge knuckles 252 of the hinge mechanism 250 spaced apart by gaps 254. The series of hinge knuckles 252 inter-fit with adjacent gaps on the second body portion of the device, and the gaps 254 receive adjacent hinge knuckles on the second body portion. The first body portion 210a also includes a first side 270a of the end piece used to bend a flexible appendage.

As best shown in the side view of FIG. 6D, the first body portion 210a defines a first inner surface 220a of the internal bore that accommodates the appendage. The first inner surface 220a preferably tapers along the length of the body portion 210a. For use on the tail of cattle, for example, the inner surface 220a preferably tapers approximately 2.5 degrees measured from the first end 222 to the second end 224. Accordingly, the first end 222 disposing adjacent the base of the tail has a larger dimension than the second end 224. The tapering first inner surface 220a in combination with a tapering second inner surface of the second body portion gives the bore a frusto-conical shape. The frusto-conical bore helps to hold the device on the preferred location on a tail, which becomes slender from the base to the tip.

As best shown in FIG. 6D, the first inner surface 220a of the bore is lined with a first gripping surface 230a. The gripping surface 230a includes a series of studs to engage or grip the surface of the appendage. When used on the base of a tail, the studs preferably protrude approximately 1/8 of an inch from the inner surface 220a and include a 35-degree draft angle. In another embodiment, the surface of the bore can include the use of a wire fillet (not shown) or glue adhesive (not shown) to make contact with the hair of skin of an animal.

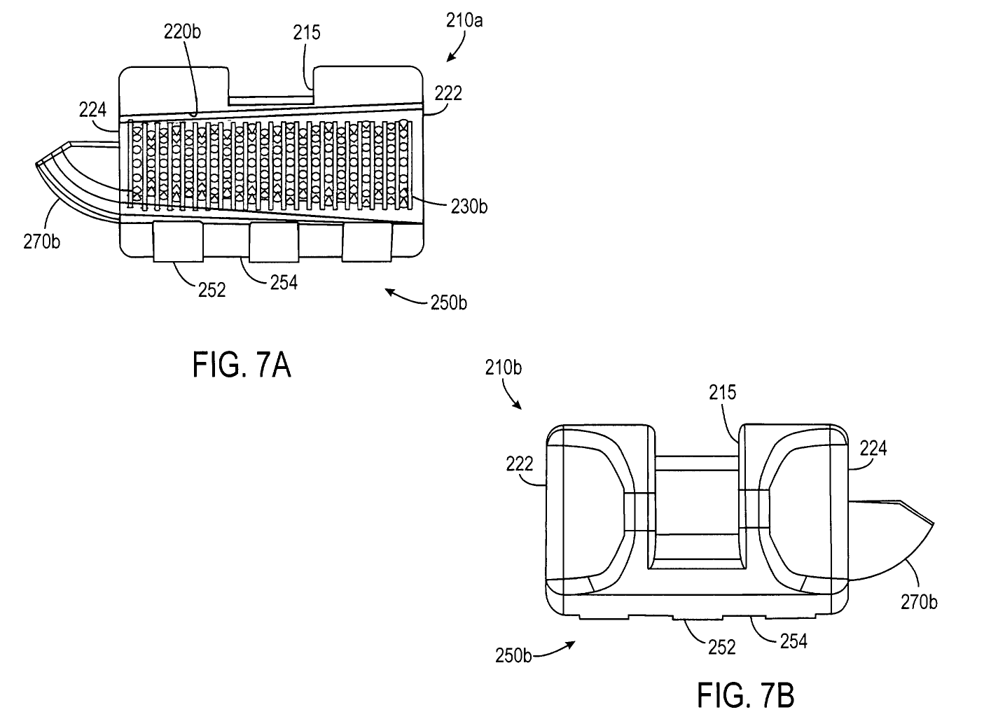

Referring to FIGS. 7A-B, the second body portion 210b of the attachment device is illustrated in first and second side views. As best shown in FIG. 7A, the second body portion 210b defines another inner surface 220b of the bore that accommodates the appendage. As before, the second inner surface 220b preferably tapers measured from the first end 222 to the second end 224. The second inner surface 220b of the bore is lined with a second gripping surface 230b, such as described above. The second body portion 210b defines the well 215 for use with the fastening mechanism (not shown). The second body portion 210b also include a second side 270b of the end piece.

Referring to FIG. 8A, components of the fastening or binding mechanism 260 of FIGS. 4A-B are illustrated. The fastening or binding mechanism 260 includes a latch or hasp 261 and an adjustable strap 264 for fastening or binding the body portions (not shown) of the device together. The latch and strap mechanism is known in the art and can be found, for example, used on a ski boot to fasten the boot on a wearer. The latch 261 includes a hinge 262, which is spring biased and attached to the second body portion. The latch 261 includes a tooth or catch 263 to engage the strap 264.

The strap 264 includes a first opening to attach the strap 264 to the first body portion. The strap 264 also includes a plurality of notches or slots 268, which are used to adjustably engage the tooth 263 of the latch 261. Preferably, numerous notches or slots 268 are included on the strap 264 and are spaced close together to provide for greater adjustment of the fastening mechanism 260. Using the adjustable fastening mechanism 260, an operator can adjust the dimension of the internal bore between the body portions and adjust the degree of clamping produced on the appendage.

Referring to FIG. 8B, another embodiment of a fastening or binding mechanism 270 for fastening or binding the two body portions (not shown) of the device together is illustrated. The fastening or binding mechanism 270 is a pull hoist mechanism with a ratchet-handle, such as is known in the art and is commonly called a COME-ALONG. The fastening or binding mechanism 270 includes a ratcheting mechanism 272, a handle 274, and an interconnecting member 276.

The handle 274 is connected to a sheave (not shown) within the ratcheting mechanism 272. The interconnecting member 276 is disposed in the ratcheting mechanism 272. The handle 274 is used by an operator to produce a ratcheting movement of the sheave. The ratcheting movement of the sheave advances the interconnecting member 276 through the ratcheting mechanism 272.

The interconnecting member 276 can be a strap, belt, chain, or the like. A first end 277 of the interconnecting member 276 is attached to one of the body portions of the device. In one embodiment, the ratcheting mechanism 272 is fixedly attached to the other body portion, and a second end 278 of the interconnecting member 276 is left free. Alternatively, the second end 278 of the interconnecting member 276 can be attached to the other body portion so that the ratcheting mechanism 272 is held between the attached ends 277 and 278. Using the handle 274, an operator can bring the body portions of the device together. The ratcheting mechanism 272 allows for fine adjustment in fastening or binding the device on an appendage. The ratcheting mechanism 272 can include a release allowing for easy unfastening of the fastening mechanism 270.

Referring to FIG. 8C, yet another embodiment of a fastening or binding mechanism 280 for fastening or binding the two body portions (not shown) of the device together is illustrated. The fastening or binding mechanism 280 resembles a fastener for footwear, such as is known in the art and is commonly used on ski boots or in-line skates. The fastening or binding mechanism 280 includes a toothed strap 282, a stirrup 284, and lever arm 288.

The strap 282 has one end 283 secured to one of the body portions (not shown). The end 283 of the strap 282 can be secured to the one body portion by a rivet, hinge, or other technique known in the art. The stirrup 284 has a pawl 286 pivotally mounted between sides of the stirrup 284 by a pin. The pawl 286 is preferably biased by a leaf or torsion spring (not shown). The toothed strap 282 is inserted between the bottom of stirrup 284 and the pawl 286 with the pawl 286 sliding and skipping on the ratchet teeth of the strap 282. The end of the pawl 286 is capable of catching on the ratchet teeth of the strap 282 to positively lock the strap 282 from being removed from the stirrup 284. By depressing the end of the pawl 286, the strap 282 can be released.

A tension lever 288 is fulcrumed on the stirrup 288. The lever 288 is also fulcrumed by a member 289, which connects to the second body portion (not shown). After ratcheting the strap 282 in the pawl 286 and stirrup 284 to a desired position, the user can further close or bind the body portions together by pivoting the tension lever 288 until it engages the toothed strap 282. The lever 288 can snap lock in the engaged position to prevent accidental loosening.

Referring to FIG. 8D, another embodiment of a fastening mechanism 290 can include a strip 292 of double-surfaced VELCRO having one end 293 attached to one of the body portions (not shown). The one end 293 has a first surface of VELCRO. A free end 294 of the strip 292 passes through a ring 298 attached to the other body portion (not shown). This other end 294 has a second surface of VELCRO, which is attachable to the first surface on the one end 293. A user can pull the strip 292 through the ring 298 in direction P until a desired fit is made on the appendage. The strip 292 can then be secured in direction S to itself by mating the opposite surfaces of the VELCRO to hold the ring 298 within a loop 296 formed with the strip 292.

As an additional example in FIG. 8E, the fastening or binding mechanism can include a self-locking cable tie 299, which is often referred to as a “wire tie.” The wire tie can be looped through opening on both sides of the hinged, attachment device and can then be tightened to secure the device on the appendage. With the benefit of the above examples and the present disclosure, one of ordinary skill in the art will appreciate that a number of fastening or binding mechanisms known in the art can be used with the attachment device 300 of the present invention.

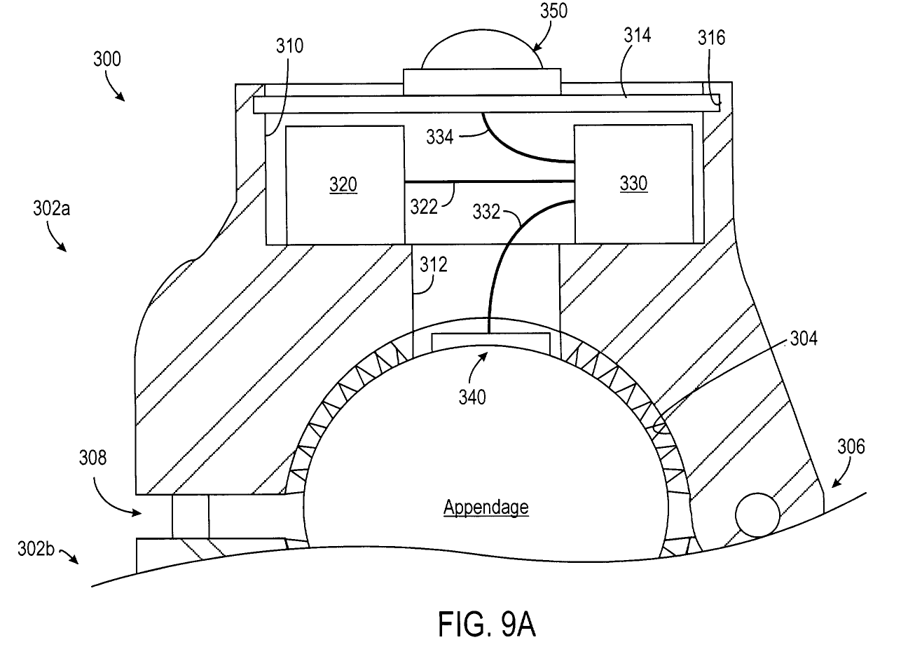

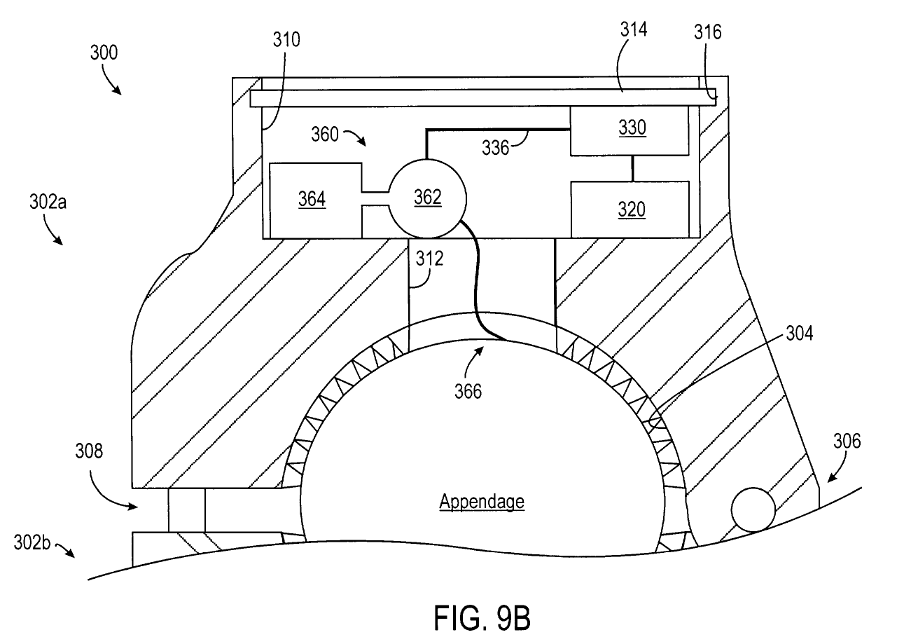

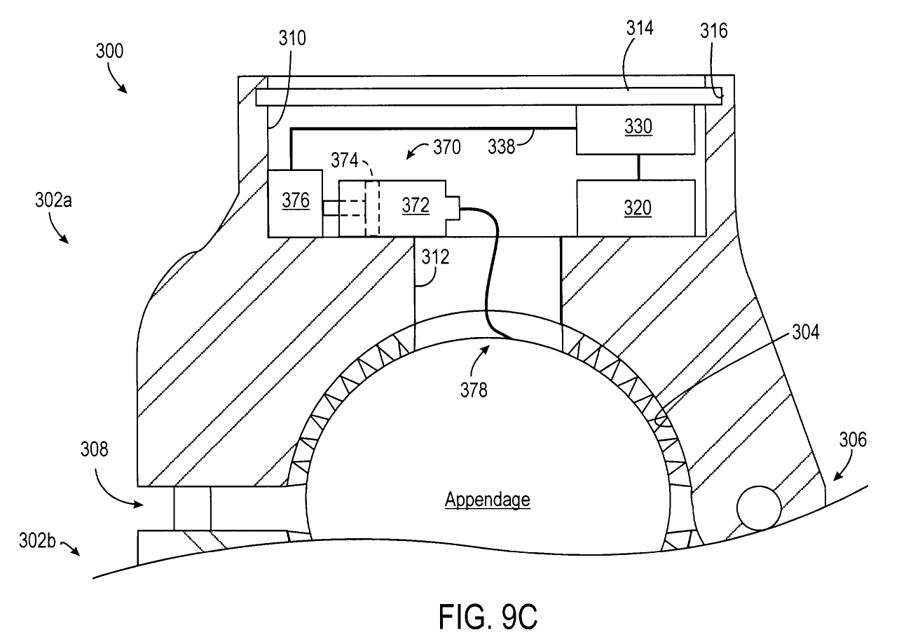

Referring to FIGS. 9A-C, embodiments of electronics and equipment for use with an attachment device 300 are illustrated. The attachment device 300 is only partially illustrated in cross-section. The attachment device 300 includes a first body portion 302a connected to a second body portion 302b by a hinge mechanism 306. The body portions 302a and 302b define an internal bore 304 accommodating an appendage. A fastening mechanism 308 fastens the body portions 302a and 302b about the appendage. The first body portion 302a defines a cavity 310 communicating with the internal bore 304 and appendage through a passage 312. A cover 314 is positioned in a guide 316 to cover the cavity 310.

The attachment device 300 of the present invention is capable of housing and protecting various types of equipment and electronics on the appendage of the animal. Furthermore, the attachment device 300 of the present invention is capable of holding a substantial amount of equipment and electronics on the appendage of the animal. The equipment or electronics are schematically illustrated and can include a microprocessor, a biosensor, a transmitter, a receiver, a tag-based system, or a global positioning system (GPS), among other possible equipment and electronics for monitoring animals, such as disclosed in U.S. Pat. No. 6,113,539 incorporated herein by reference in its entirety. The equipment and electronics can be separate components installed in the cavity 310 or attached to the device 300. Alternatively, the equipment and electronics can be a self-contained instrument pack that is disposed in the cavity 310 of the device 300.

In one embodiment illustrated in FIG. 9A, the electronics and equipment for use with the attachment device 300 include a power supply 320, a microprocessor 330, a biosensor 340, and an indicator 350. The power supply 320 and microprocessor 330 are housed in the cavity 310 of the first body portion 302a. The power supply 320, such as a battery, is electrically coupled to the microprocessor 330 via a connection 322 and supplies power thereto.

The biosensor 340 is positioned adjacent the surface of the animal's appendage held within the bore 304. The biosensor 340 senses physiological parameters of the animal and electrically couples to the microprocessor 330 via a connection 332 that passes through a passage 312. The microprocessor 330 processes the sensed parameters from the biosensor 340. The microprocessor 330 is electrically coupled to the indicator 350 via a connection 334. The indicator 350 is attached to a cover 314 and can include a visual or auditory alarm, such as a strobe light, beacon, or piezoelectric horn, among other possibilities known in the art.

When a monitored physiological parameter of the animal is out of a predetermined “healthy” range, the microprocessor 330 can activate the alarm 350 to alert a feedlot operator of a potential health problem for that particular animal. In other embodiments, the microprocessor 330 can be coupled to a transmitter (not shown) for transmitting the sensed physiological parameter or alarm to a remote station. In addition, the microprocessor 330 can be coupled to a GPS-system or a tag-based system (not shown) to track the location, movement, feeding habits, and watering habits of the animal.

In another embodiment illustrated in FIG. 9B, the electronics or equipment for use with the attachment device 300 includes a power supply 320, a microprocessor 330, and administering or injection equipment 360. The administering or injection equipment 360 includes an actuator or miniature pump 362, a reservoir 364, and an intravenous line or catheter 366.

The power supply 320 is electrically coupled to the microprocessor 330 and supplies power thereto. The microprocessor 330 is electrically coupled to the miniature pump 362 via a connection 336. The miniature pump 362 is coupled to the reservoir 364, which contains a substance to be administered to the animal. The substance can include, for example, antibiotics, vaccines, hormones, steroids, medicines, blood products, chemicals, etc. The intravenous line 366 is coupled to the pump 362 and passes through the passage 312 to the appendage of the animal.

In one embodiment, for example, the microprocessor 330 can include a timing circuit or programming that activates the miniature pump 362 according to predetermined time intervals or timing scheme. In another embodiment, the microprocessor 330 can be electrically coupled to a receiver (not shown) for receiving a remote signal to activate the pump 362. The microprocessor 330 activates the pump 362. The miniature pump 362 pumps at least a portion of the substance from the reservoir 364 and delivers it to the animal via the intravenous line 366 coupled to the appendage.

In yet another embodiment illustrated in FIG. 9C, the electronics or equipment for use with the attachment device 300 includes a power supply 320, a microprocessor 330, and administering or injection equipment 370. The administering or injection equipment 370 includes a reservoir 372 and an actuator 376. The actuator 376 is a linear actuator or servo moving a plunger 374 disposed in the reservoir 372.

The reservoir 372 is coupled to an intravenous line 378. The actuator 376 is operatively coupled to the microprocessor 330 via a connection 338. The microprocessor 330 activates the actuator 376, which moves the plunger 374. The plunger 374 pushes the substance in the reservoir 372 and delivers it to the animal via the intravenous line 378 coupled to the appendage.

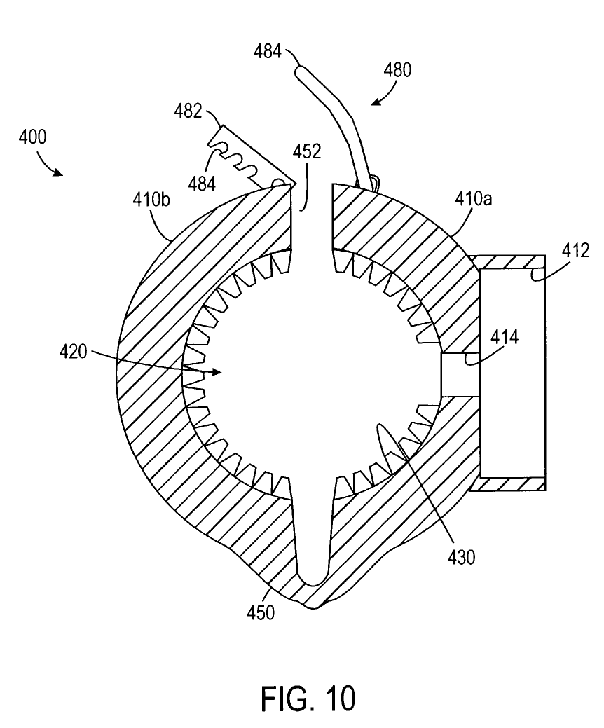

Referring to FIG. 10, another embodiment of an attachment device 400 is illustrated in cross-sectional. The attachment device 400 includes a first body portion 410a, a second body portion 410b, a hinge mechanism 450, and a fastening mechanism 480. The attachment device 400 can also include an end piece (not shown) as discussed above. The body portions 410a and 410b define inner surfaces of a bore 420 for accommodating an appendage (not shown) of the animal to be monitored. The first body portion 410a defines a cavity 412 for holding equipment or electronics (not shown). A passage 414 communicates the cavity 412 with the internal bore 420.

The body portions 410a and 410b are attached together by the hinge mechanism 450. In the present embodiment, the hinge mechanism 450 is a thin fold of material integrally connecting the rigid body portions 410a and 410b together along one edge. The hinge mechanism 450 allows the body portions 410a and 410b to open and close relative to one another. The hinge mechanism 450 can be biased like a leaf spring. Furthermore, the hinge mechanism 450 can produce a gap 452 along the free edges of the body portions 410a and 410b as discussed above.

The fastening mechanism 480 maintains the unconnected edges of the body portions 410a and 410b together and holds the device 400 on the appendage of the animal. In the present embodiment of the invention, the fastening mechanism 480 includes a latch 482 and a loop 484. The latch 482 is hingedly attached to the second body portion 410b. The loop 484 is hingedly attached to the first body portion 410a. The latch 482 includes notches 483, which receive the loop 484. The notches 483 allow an operator to adjust the dimension of the bore 420 and the degree of clamping produced on the appendage by the device 400.

While the invention has been described with reference to the preferred embodiments, obvious modifications and alterations are possible by those skilled in the related art. Therefore, it is intended that the invention include all such modifications and alterations to the full extent that they come within the scope of the following claims or the equivalents thereof.

What is claimed is:

1. A device for attaching equipment to an appendage comprising:
a first portion;
a second portion disposed adjacent the first portion; the first and second portions defining a bore therebetween for the appendage;
a hinge mechanism connecting the first portion to the second portion and enabling the portions to open and close about the appendage disposed in the bore; and
a second mechanism fastening the portions together about the appendage disposed in the bore.

2. The device of claim 1, wherein one of the portions defines a cavity communicating with the bore and housing the equipment.

3. The device of claim 1, wherein the hinge mechanism comprises a plurality of knuckles connecting the first and second portions along adjacent edges and pivoting on a pin.

4. The device of claim 1, wherein the hinge mechanism comprises a flexible member connecting the first and second portions along adjacent edges.

5. The device of claim 1, wherein the bore has a first end and a second end, the first end having a first dimension greater than a second dimension of the second end.

6. The device of claim 1, further comprising an end piece attached to the device adjacent an end of the bore and being curved in relation to the bore.

7. The device of claim 1, wherein the second mechanism is adjustable.

8. The device of claim 1, wherein the bore has a series of studs disposed thereon.

9. A device for attaching equipment to an appendage comprising:
a first portion;
a second portion disposed adjacent the first portion; the portions defining a bore therebetween for the appendage;
a surface disposed on the bore and engaging the appendage disposed in the bore;
a hinge mechanism connecting the first portion to the second portion along adjacent edges and enabling the portions to open and close about the appendage disposed in the bore; and
a second mechanism fastening the portions together about the appendage disposed in the bore.

10. The device of claim 9, further comprising an end piece attached to one end of the device adjacent the bore and being curved in relation to the bore.

11. The device of claim 9, wherein one of the portions defines a cavity communicating with the bore and housing the equipment.

12. The device of claim 9, wherein the bore is frusto-conical.

13. The device of claim 9, wherein the hinge mechanism comprises a plurality of knuckles inter-fitting together and pivoting on a pin.

14. The device of claim 9, wherein the second mechanism comprises:
an interconnecting member attached to the first portion and having a plurality of slots;
a latch hingedly attached to the second portion and adjustably engaging one of the slots.

15. The device of claim 9, wherein the second mechanism comprises:
an interconnecting member attached to the first portion; and
a ratcheting mechanism attached to the second member and capable of moving the interconnecting member to fasten the portions together.

16. The device of claim 9, wherein the second mechanism comprises:
a strap attached to the first portion and having a plurality of teeth;
a stirrup receiving the strap therein;
a releasable pawl in the stirrup ratcheting on the teeth when inserted in one direction and engaging on the teeth in an opposite direction; and
a fulcrumed lever attached to the stirrup and the second portion, the lever engaging the strap when locking the second mechanism.

17. The device of claim 9, wherein the surface comprises a plurality of studs disposed on the bore.

18. A device for attaching equipment to an appendage comprising:
means for holding the equipment on the device;
means for enclosing the device about the appendage;
means for engaging a surface of the appendage; and
means for fastening the enclosing means on the appendage.

19. The device of claim 18, further comprising means for bending the appendage with the device.

20. The device of claim 19, wherein the bending means comprises a curved end piece attached to an end of the device.

21. The device of claim 18, wherein the holding means comprises means for protecting the equipment.

22. The device of claim 21, wherein the holding means comprises a cavity defined in the device and having a cover.

23. The device of claim 18, wherein the enclosing means comprises a hinge mechanism attaching a first portion to a second portion of the device along adjacent edges.

24. The device of claim 18, wherein the engaging means comprises a series of studs disposed on an inner bore engaging a surface of the appendage.

25. The device of claim 18, wherein the fastening means further comprises means for clamping the device about the appendage.

26. The device of claim 18, wherein the fastening means further comprises means for adjusting the enclosing means about the appendage.

27. The device of claim 26, further comprising means for ratcheting the adjustable means.

28. The device of claim 26, wherein the fastening means comprises:
an interconnecting member attached to a first portion of the device and having a plurality of slots, and
a latch hingedly attached to a second portion of the device and adjustably connecting with the slots of the interconnecting member.

29. The device of claim 26, wherein the second mechanism comprises:
an interconnecting member attached to the first portion; and
a ratcheting mechanism attached to the second member and capable of moving the interconnecting member to fasten the portions together.

30. The device of claim 26, wherein the second mechanism comprises:
a strap attached to the first portion and having a plurality of teeth;
a stirrup receiving the strap therein;
a releasable pawl in the stirrup ratcheting on the teeth when inserted in one direction and engaging on the teeth in an opposite direction; and
a fulcrumed lever attached to the stirrup and the second portion, the lever engaging the strap when locking the second mechanism.

31. A device for administering a substance to an animal having an appendage comprising:
a body capable of being removably attached to the appendage and defining a cavity therein communicating with the appendage;
a reservoir disposed in the cavity and containing the substance;
an actuator disposed in the cavity and coupled to the reservoir; and
a microprocessor disposed in the cavity and activating the actuator to deliver at least a portion of the substance from the reservoir to the animal through a line.

32. The device of claim 31, wherein the actuator comprises a miniature pump coupled to the reservoir and the line.

33. The device of claim 31, wherein the actuator comprises a servo moving a plunger disposed in the reservoir.

34. The device of claim 31, wherein the substance is selected from the group consisting of an antibiotic, a vaccine, a steroid, a medicine, a chemical, or a hormone.

35. The device of claim 31, wherein the microprocessor automatically activates the actuator based on a timing scheme.

36. The device of claim 31, wherein the microprocessor is electrically coupled to a receiver for receiving a remote signal to activate the actuator.

37. The device of claim 31, wherein the body comprises:
a first portion;
a second portion hingedly attached to the first portion; the portions defining a bore therebetween for the appendage, at least one of the portions defining the cavity communicating with the bore; and
a mechanism fastening the portions together about the appendage disposed in the bore.
